(19) United States
(12) Patent Application Publication
(10) Pub. No.: US 2007/0240236 A1
(43) Pub. Date: Oct. 11, 2007

(54) O-GLYCANS IN THE TREATMENT OF INFLAMMATORY BOWEL DISEASE AND CANCERS

(75) Inventor: Lijun Xia, Edmond, OK (US)

Correspondence Address: FULBRIGHT & JAWORSKI L.L.P., 600 CONGRESS AVE., SUITE 2400, AUSTIN, TX, 78701, US

(73) Assignee: OKLAHOMA MEDICAL RESEARCH FOUNDATION

(21) Appl. No.: 11/696,599

(22) Filed: Apr. 4, 2007

Related U.S. Application Data
(60) Provisional application No. 60/789,499, filed on Apr. 5, 2006.

(51) Int. Cl. A01K 67/027; A61K 38/17; A61K 31/715

(52) U.S. Cl. 800/18; 514/54; 514/8

(57) ABSTRACT

The present invention provides preventive approaches and treatments for an inflammatory bowel disorder (e.g., Crohn's disease or ulcerative colitis) or a gastrointestinal tumor (e.g., a colorectal cancer) comprising administering an O-glycan composition (e.g., mucins) to a subject, such as a human patient. In addition, the present invention also provides transgenic mice that fail to synthesize core 1-derived or core 3-derived O-glycans.

This application claims benefit of priority to U.S. Provisional Application Ser. No. 60/789,499, filed Apr. 5, 2006, the entire contents of which are hereby incorporated by reference.

The government owns rights in the present invention pursuant to grant number P20-RR018758 from the National Institutes of Health.

### BACKGROUND OF THE INVENTION

1. Field of the Invention

The present invention relates generally to the fields of glycobiology and medicine. More particularly, it concerns use of an O-glycan composition (e.g., mucins) to prevent or treat inflammatory bowel diseases (e.g., Crohn's disease and ulcerative colitis) or gastrointestinal tumors.

2. Description of Related Art

Ulcerative colitis is a common form of inflammatory bowel diseases (IBD). It is generally recognized as an immune-mediated disorder resulting from an abnormal interaction between colonic microflora and mucosal immune cells in a genetically susceptible host (Sartor, 2003; Podolsky, 2002; Elson et al., 2003). The nature of the mucosal immune abnormality remains unclear, and how this interaction is allowed to develop is not well understood. In addition, all drugs currently on the market for the treatment of ulcerative colitis have side effects and none of them can cure the disease. In addition, IBD has been associated with an increased risk of colorectal cancer (Dixon et al., 2006).

Altered intestinal O-glycan expression has long been observed in patients with IBD and colorectal cancer, but the role of this alteration in the etiology of these diseases is unknown (Corfield et al., 2001; Rhodes, 1997; Podolsky and Isselbacher, 1984). Thus, there a need for additional understanding of the role O-glycans play in the development of these disease states.

### SUMMARY OF THE INVENTION

The present invention overcomes deficiencies in the prior art by providing methods for treating an IBD comprising administering an O-glycan composition (e.g., mucins) to a subject (e.g., a human patient). Additionally, the present invention also provides methods for preventing and treating a gastrointestinal cancer (e.g., colorectal cancer) comprising administering an O-glycan composition (e.g., mucins) to a subject, such as a human patient.

Thus, in accordance with the present invention, there is provided a method of preventing development of or treating an inflammatory bowel disease comprising administering to a subject in need thereof an O-glycan composition. The inflammatory bowel disease may be ulcerative colitis or Crohn's disease. The O-glycan composition may comprise a mucin composition, for example, including one or more of Muc1, Muc2, Muc3, Muc4, Muc5AC, Muc6, or Muc13. The method may further comprise administering to said subject a second therapeutic composition, such as an anti-inflammatory agent or an antibiotic.

The O-glycan composition may be formulated for release in the small intestine, for example, in the ileum, jejunum or duodenum. It also may be formulated for release in the large intestine, for example, in the cecum, ascending colon, transverse colon, descending colon, sigmoid colon or rectum. The subject may be a mammal, e.g., a human. The mucin composition may comprise mucins obtained from a mammal, either human or non-human. The mucins may be purified by centrifugation, treated with DNAse, RNAse, protease and lipase. The mucins may be further purified by chromatography. The mucins may be derived from stomach or colon. Alternatively, the mucins may be recombinantly expressed in a mammalian expression system.

In another embodiment, there is provided a transgenic mouse with functional T-synthase gene flanked with Lox P sites. In yet another embodiment, there is provided a transgenic mouse lacking any functional T-synthase gene in intestinal epithelial cells. In still yet another embodiment, there is provided a transgenic mouse having one functional and one non-function T-synthase gene in intestinal epithelial cells. In a further embodiment, there is provided a transgenic mouse lacking any functional core 3β1,3-N-acetylglucosaminyltransferase gene. In still a further embodiment, there is provided a transgenic mouse having one functional and one non-function core 3β1,3-N-acetylglucosaminyltransferase gene. In still yet a further embodiment, there is provided a transgenic mouse lacking any functional T-synthase gene in intestinal epithelial cells and any functional core 3β1,3-N-acetylglucosaminyltransferase. In still another embodiment, there is provided a transgenic mouse having and one functional and one non-functional T-synthase gene in intestinal epithelial cells, and one functional and one non-functional core 3β1,3-N-acetylglucosaminyltransferase gene.

In yet another embodiment, there is provided a method of preventing development of colorectal tumor comprising administering to a subject in need thereof an O-glycan composition. The colorectal tumor may be a colorectal adenomatous polyp, a colorectal adenoma, or a colorectal carcinoma. The O-glycan composition comprises a mucin composition, for example, a mucin composition comprising one or more of Muc1, Muc2, Muc3, Muc4, Muc5AC, Muc6, or Muc13. The method may further comprise administering to said subject a second therapy, such as an anti-inflammatory agent or an antibiotic.

The O-glycan composition may be formulated for release in the small intestine, for example, in the ileum, jejunum or duodenum. It also may be formulated for release in the stomach and large intestine, for example, in the cecum, ascending colon, transverse colon, descending colon, sigmoid colon or rectum. The subject may be a mammal, e.g., a human. The mucin composition may comprise mucins obtained from a mammal, either human or non-human. The mucins may be purified by centrifugation, treated with DNAse, RNAse, protease and lipase. The mucins may be further purified by chromatography. The mucins may be derived from stomach or colon. Alternatively, the mucins may be recombinantly expressed in a mammalian expression system.

In still an additional embodiment, there is provided a pharmaceutical composition comprising an O-glycan composition dispersed in a pharmaceutically acceptable buffer, diluent or excipient. The O-glycan composition may comprise a mucin composition, such as one or more of Muc1, Muc2, Muc3, Muc4, Muc5AC, Muc6, or Muc13. The O-glycan composition may be formulated for release in the stomach or small intestine, for example, in the ileum, jejunum or duodenum. It also may be formulated for release in the large intestine, for example, in the cecum, ascending colon, transverse colon, descending colon, sigmoid colon or rectum.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—The scheme shows the two main types of O-glycan core structures. T-synthase refers to core 1β1,3-galactosyltransferase. C3GnT refers to core 3β1,3-N-acetylglucosaminyltransferase. Arrowheads show the possible pathways for further branching, elongation, fucosylation, sialylation and sulfation.

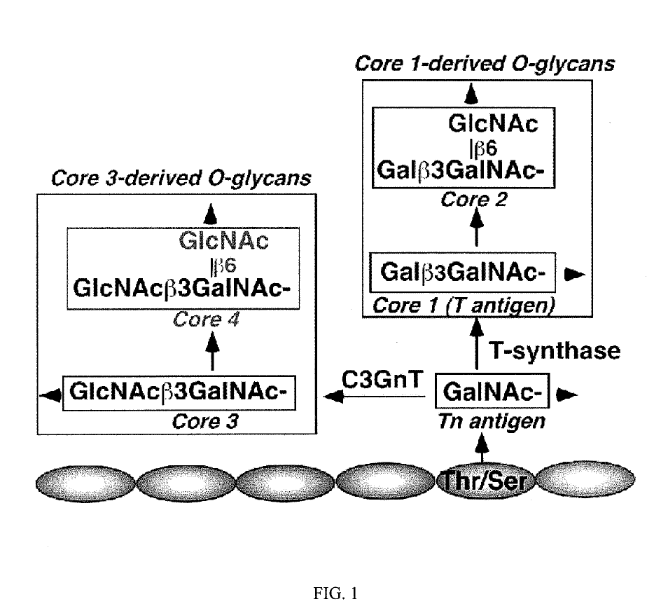

FIGS. 2A-E—Mice engineered to lack core 3-derived O-glycans by targeting the gene for C3GnT (C3GnT^{-/-}). (FIG. 2A) Gene targeting strategy. (FIG. 2B) Southern blot genotyping. (FIG. 2C) RT-PCR confirms the deletion of C3GnT gene product. (FIG. 2D) Enzymatic assay shows the elimination of the C3GnT activity in C3GnT-deficient tissues. (FIG. 2E) LacZ staining confirms the C3GnT expression pattern.

FIGS. 3A-D—(FIGS. 3A-B) C3GnT^{-/-} colon has reduced carbohydrate expression. (FIGS. 3C-D) Comparison of body changes (FIG. 3C) and inflammation in HE-stained colonic tissues (FIG. 3D) of C3GnT^{-/-} and WT mice seven days after DSS treatment.

FIGS. 4A-B—(FIG. 4A) C3GnT^{-/-} more susceptible to DSS and AOM-induced colorectal tumor (*). (FIG. 4B) Histology shows the in situ adenocarcinoma (arrowhead).

FIGS. 5A-D—Generation of mice lacking T-synthase gene specifically in intestinal epithelial cells (Epi T-syn^{-/-}). (FIG. 5A) Strategy for generation of Epi T-syn^{-/-} mice. (FIG. 5B) PCR™ genotyping of DNA isolated from tail tissue. (FIG. 5C) T-syn mRNA was completely abolished in T-syn^{-/-} intestinal epithelial cells. (FIG. 5D) Epi T-syn^{-/-} but not WT intestinal epithelium was specifically stained positive for Tn antigens.

FIGS. 6A-F—(FIG. 6A) Growth curves of WT and Epi T-syn^{-/-} mailes. (FIG. 6B) An Epi T-syn^{-/-} mouse exhibited rectal prolapse. (FIG. 6C) The large intestines from 20-week-old WT and Epi T-syn^{-/-} mice. (FIG. 6D) MLNs from an Epi T-syn^{-/-} mouse compared with that from a WT control. (FIG. 6E-F) HE-stained WT and Epi T-syn^{-/-} distal colon sections.

FIGS. 7A-B—Representative images of PAS and Muc2 staining of colonic sections.

FIGS. 8A-E—(FIG. 8A) Breeding strategy for generation of Epi T-syn^{-/-} and C3GnT^{-/-} double knockout mice (DKO). (FIG. 8B) Anti-TN mAb staining. (FIG. 8C) Western blot with a Tn-specific lectin, HPA. β-actin was used as a loading control. (FIG. 8D-E) Growth curves (FIG. 8D) and colonic histology (FIG. 8E; 6-wks old) of WT and DKO males.

FIGS. 9A-D—(FIG. 9A) Growth curves of mucin-treated or sham-treated Epi T-syn^{-/-} mice. (FIG. 9B) HE-stained WT colonic tissue as a histology control. (FIGS. 9C-D) Representative of HE-stained colonic tissues of Epi T-syn^{-/-} mice seven weeks after being treated with or without mucins.

FIGS. 10A-B—Mucins were prepared by collecting luminal surface layer of the porcine colon followed by alcohol precipitation. Products were run on SDS-PAGE gels. (FIG. 9A) PAS staining showing glycans. (FIG. 9B) Coomassie staining testing protein contamination Fraction 4 was the final product and used for experiments.

FIG. 11-Mucin therapeutic trial with porcine colon mucins. WT or C3GnT−/−-Epi Tsyn^{-/-} mice (three in each group) with colitis were treated with porcine colon mucins or albumin control, Body weight changes compared to baseline.

### DESCRIPTION OF ILLUSTRATIVE EMBODIMENTS

O-glycans are primary components of the intestinal mucus gel layer that overlies the gut epithelium. This layer is a dense polysaccharide-rich matrix that, together with epithelial cells, composes the intestinal barrier, which functions to prevent intestinal microflora from encountering intestinal mucosal immune cells. The mucus layer consists primarily of mucins, molecules rich in serine and threonine to which O-linked oligosaccharides (O-glycans) are frequently attached. Over 80% of mucin mass consists of O-glycans. O-glycans have two main subtypes referred to as core 1- and core 3-derived O-glycans, and the biosynthesis of these subtypes is controlled by specific glycosyltransferases.

To address the roles of O-glycans in intestinal function in vivo, in the pathogenesis of inflammatory bowel disease (IBD), and gastrointestinal cancer, the inventors established a mouse line that is deficient in corel-derived O-glycans. They also have also developed a line that is deficient in core 3-derived O-glycans, and a line that is deficient in both. By using these mouse lines, the inventors have been able to identify specific contributions to the aforementioned disease states and, moreover, to alleviate symptoms of these disease states by administration of O-glycans to subjects.

Thus, the present invention demonstrates that alteration of the function of certain O-glycans can result in profound phenotypes in vivo. The present invention provides methods for preventing and treating an IBD or preventing a gastrointestinal cancer (e.g., a colorectal cancer) comprising administering an O-glycan (e.g., a mucin) to a subject (e.g., a human patient).

### I. O-GLYCANS

Glycoproteins with O-glycosidically linked carbohydrate chains of complex structures and functions are found in secretions and on the cell surfaces of cancer cells. The structures of O-glycans are often unusual or abnormal in cancer, and greatly contribute to the phenotype and biology of cancer cells. Some of the mechanisms of changes in O-glycosylation pathways have been determined in cancer model systems. However, O-glycan biosynthesis is a complex process. The glycosyltransferases that synthesize O-glycans appear to exist as families of related enzymes of which individual members are expressed in a tissue- and growth-specific fashion. Studies of their regulation in cancer may reveal the connection between cancerous transformation and glycosylation which may help to understand and control the abnormal biology of tumor cells. Cancer diagnosis may be based on the appearance of certain glycosylated epitopes, and therapeutic avenues have been designed to attack cancer cells via their glycans.

A. Mucins

Mucins are high-molecular weight epithelial glycoproteins with a high content of clustered oligosaccharides O-glycosidically linked to tandem repeat peptides rich in threonine, serine, and proline. There are two structurally and functionally distinct classes of mucins: secreted gel-forming mucins (MUC2, MUC5AC, MUC5B, and MUC6) and transmembrane mucins (MUC1, MUC3A, MUC3B, MUC4, MUC12, MUC17), although the products of some MUC genes do not fit well into either class (MUC7, MUC8, MUC9, MUC13, MUC15, MUC16). MUC1 mucin, as detected immunologically, is increased in expression in colon cancers, which correlates with a worse prognosis. Expression of MUC2 secreted gel-forming mucin is generally decreased in colorectal adenocarcinoma, but preserved in mucinous carcinomas, a distinct subtype of colon cancer associated with microsatellite instability. Another secreted gel-forming mucin, MUC5AC, a product of normal gastric mucosa, is absent from normal colon, but frequently present in colorectal adenomas and colon cancers. The O-glycosidically linked oligosaccharides of mucins can be described in terms of core type, backbone type, and peripheral structures.

B. O-glycan Pharmaceutical Preparations

Pharmaceutical compositions of the present invention comprise an effective amount of O-glycans or mucins dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one O-glycan or additional active ingredient will be known to those of skill in the art in light of the present disclosure, as exemplified by Remington's Pharmaceutical Sciences, 18^{th} Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered orally, or rectally, but may also be administered intratracheally, intranasally, subcutaneously, mucosally, locally, inhalation (e.g., aerosol inhalation), injection, infusion, continuous infusion, localized perfusion bathing target cells directly, via a catheter, via a lavage, or by other method or any combination of the foregoing as would be known to one of ordinary skill in the art (see, for example, Remington's Pharmaceutical Sciences, 18^{th} Ed. Mack Printing Company, 1990, incorporated herein by reference).

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compounds of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In particular embodiments, the O-glycan compositions of the present invention are prepared for administration by such routes as oral ingestion. In these embodiments, the solid composition may comprise, for example, solutions, suspensions, emulsions, tablets, pills, capsules (e.g., hard or soft shelled gelatin capsules), delayed release capsules, sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain specific embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof, a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum. After insertion, suppositories soften, melt or dissolve in the cavity. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

C. O-glycan Production and Purification

O-glycans of the present invention may be purified from an animal, or a recombinant O-glycan may be generated and optionally purified. In certain embodiments, a recombinant human O-glycan may be expressed in cells and subsequently purified (e.g., using centrifugation and/or chromatography).

Generally, for purification, the inventors will follow published methods for purification of mucins from fresh porcine stomach or colon with modifications. Xia et al. (2005); Feste et al. (1990). Briefly, after removing contents and briefly rinsing in water, the mucosal layer (including epithelium and mucus) of porcine stomach or colon is removed by scraping. The mucosal material is homogenized in ice-cold water (˜1 part mucosa: 1 part water, final slurry), and centrifuged to remove insoluble debris. The soluble mucins in the supernatant are precipitated by adjusting to pH 5.0 with 100 mM HCl followed by centrifugation (10,000×g, 4° C., 10 min). The pellet is resolubilized and adjusted to pH 7.2 with 100 mM NaOH, then extracted twice in methanol:chloroform (1:1 v/v) prior to a second centrifugation. The middle phase is collected and dialyzed (12-14,000 MWCO) followed by sequential treatment with heparinum Heparinase II (0.075 U/ml, Sigma), chondroitinase ABC (0.015 U/ml, Sigma), DNase (75 U/ml, Invitrogen), RNase (0.01 mg/ml, Invitrogen), and proteinase K (0.25 U/ml, O/N at 65° C., Sigma). These treatments eliminate contaminating lipids, polypeptides, and nucleotides. The mucin is then collected as a >200 kDa void volume fraction by size exclusion chromatography (Sephacryl HR-S-200, Pharmacia) in isotonic buffer (50 mM Tris, 100 mM NaCl, pH 7.4). The void volume fraction is dialyzed, lyophilized, weighed, and stored at −80° C. The quality of the purified mucins is verified by SDS-PAGE using a 3% stacking and a 4% separating gel that is stained by PAS. Protein will be measured using a BCA kit (Pierce).

D. O-glycan Variants

As modifications and/or changes may be made in the structure of a mucin (e.g., a Muc1, Muc2, Muc3, Muc4, Muc5AC, Muc6, and/or Muc13), the present invention contemplates variation in mucins and other O-glycan composition which nonetheless retain substantial activity with respect to the preventative and curative aspects of the present invention.

1. Modified Polynucleotides and Polypeptides

The biological functional equivalent may comprise a polynucleotide that has been engineered to contain distinct sequences while at the same time retaining the capacity to encode the “wild-type” or standard protein. This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein.

In another example, a polynucleotide made be (and encode) a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) may be substituted.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

2. Altered Amino Acids

The present invention, in many aspects, relies on the synthesis of peptides and polypeptides in cyto, via transcription and translation of appropriate polynucleotides. These peptides and polypeptides will include the twenty “natural” amino acids, and post-translational modifications thereof. However, in vitro peptide synthesis permits the use of modified and/or unusual amino acids. A table of exemplary, but not limiting, modified and/or unusual amino acids is provided herein below.

TABLE 1
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta- | AHyl | allo-Hydroxylysine |
| | Amino-propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

3. Mimetics

In addition to the biological functional equivalents discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128, 5,710,245, 5,840,833 and 5,859,184. Theses structures render the peptide or protein more thermally stable, also increase resistance to proteolytic degradation. Six-, seven-, eleven-, twelve-, thirteen- and fourteen-membered ring structures are disclosed.

Methods for generating conformationally restricted beta turns and beta bulges are described, for example, in U.S. Pat. Nos. 5,440,013, 5,618,914 and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

### II. INFLAMMATORY BOWEL DISEASE

An O-glycan compound or composition of the present invention (e.g., mucins), may be used to treat an inflammatory bowel disease. In certain embodiments, the IBD treated by the present invention is ulcerative colitis or Crohn's disease. However, the term “inflammatory bowel disease” or “IBD”, as used herein, describes a broad class of diseases characterized by inflammation of at least part of the gastrointestinal tract. IBD symptoms may include inflammation of the intestine and resulting in abdominal cramping and persistent diarrhea. Inflammatory bowel diseases include ulcerative colitis (UC), Crohn's disease (CD), indeterminate colitis, chronic colitis, discontinuous or patchy disease, ileal inflammation, extracolonic inflammation, granulomatous inflammation in response to ruptured crypts, aphthous ulcers, transmural inflammation, microscopic colitis, diverticulitis and diversion colitis.

A. Ulcerative Colitis

As discussed above, altered intestinal O-glycan expression has long been observed in patients with IBD, such as ulcerative colitis, but the role of this alteration in the etiology of these diseases is unknown (Rhodes, 1996; 1997; Podolsky & Fournier, 1988). Ulcerative colitis is a disease that causes inflammation and sores, called ulcers, in the lining of the large intestine. The inflammation usually occurs in the rectum and lower part of the colon, but it may affect the entire colon. Ulcerative colitis rarely affects the small intestine except for the end section, called the terminal ileum. Ulcerative colitis may also be called colitis or proctitis. The inflammation makes the colon empty frequently, causing diarrhea. Ulcers form in places where the inflammation has killed the cells lining the colon; the ulcers bleed and produce pus.

Ulcerative colitis may occur in people of any age, but most often it starts between ages 15 and 30, or less frequently between ages 50 and 70. Children and adolescents sometimes develop the disease. Ulcerative colitis affects men and women equally and appears to run in some families. Theories about what causes ulcerative colitis abound, but none have been proven. The most popular theory is that the body's immune system reacts to a virus or a bacterium by causing ongoing inflammation in the intestinal wall. People with ulcerative colitis have abnormalities of the immune system, but doctors do not know whether these abnormalities are a cause or a result of the disease. Ulcerative colitis is not caused by emotional distress or sensitivity to certain foods or food products, but these factors may trigger symptoms in some people.

The most common symptoms of ulcerative colitis are abdominal pain and bloody diarrhea. Patients also may experience fatigue, weight loss, loss of appetite, rectal bleeding, and loss of body fluids and nutrients. About half of patients have mild symptoms. Others suffer frequent fever, bloody diarrhea, nausea, and severe abdominal cramps. Ulcerative colitis may also cause problems such as arthritis, inflammation of the eye, liver disease (hepatitis, cirrhosis, and primary sclerosing cholangitis), osteoporosis, skin rashes, and anemia. No one knows for sure why problems occur outside the colon. Scientists think these complications may occur when the immune system triggers inflammation in other parts of the body. Some of these problems go away when the colitis is treated.

A thorough physical exam and a series of tests may be required to diagnose ulcerative colitis. Blood tests may be done to check for anemia, which could indicate bleeding in the colon or rectum. Blood tests may also uncover a high white blood cell count, which is a sign of inflammation somewhere in the body. By testing a stool sample, the doctor can detect bleeding or infection in the colon or rectum. The doctor may do a colonoscopy or sigmoidoscopy. For either test, the doctor inserts an endoscope—a long, flexible, lighted tube connected to a computer and TV monitor—into the anus to see the inside of the colon and rectum. The doctor will be able to see any inflammation, bleeding, or ulcers on the colon wall. During the exam, the doctor may do a biopsy, which involves taking a sample of tissue from the lining of the colon to view with a microscope. A barium enema x-ray of the colon may also be required. This procedure involves filling the colon with barium, a chalky white solution. The barium shows up white on x-ray film, allowing the doctor a clear view of the colon, including any ulcers or other abnormalities that might be there.

Treatment for ulcerative colitis depends on the seriousness of the disease. Most people are treated with medication. In severe cases, a patient may need surgery to remove the diseased colon. Surgery is the only cure for ulcerative colitis. Some people whose symptoms are triggered by certain foods are able to control the symptoms by avoiding foods that upset their intestines, like highly seasoned foods, raw fruits and vegetables, or milk sugar (lactose). Each person may experience ulcerative colitis differently, so treatment is adjusted for each individual. Emotional and psychological support is important. Some people have remissions—periods when the symptoms go away—that last for months or even years. However, most patients' symptoms eventually return. This changing pattern of the disease means one cannot always tell when a treatment has helped. Some people with ulcerative colitis may need medical care for some time, with regular doctor visits to monitor the condition.

The goal of therapy is to induce and maintain remission, and to improve the quality of life for people with ulcerative colitis. Several types of drugs are available:
Aminosalicylates—drugs that contain 5-aminosalicyclic acid (5-ASA), help control inflammation. Sulfasalazine is a combination of sulfapyridine and 5-ASA and is used to induce and maintain remission. The sulfapyridine component carries the anti-inflammatory 5-ASA to the intestine. However, sulfapyridine may lead to side effects such as include nausea, vomiting, heartburn, diarrhea, and headache. Other 5-ASA agents such as olsalazine, mesalamine, and balsalazide, have a different carrier, offer fewer side effects, and may be used by people who cannot take sulfasalazine. 5-ASAs are given orally, through an enema, or in a suppository, depending on the location of the inflammation in the colon. Most people with mild or moderate ulcerative colitis are treated with this group of drugs first.
Corticosteroids—such as prednisone and hydrocortisone also reduce inflammation. They may be used by people who have moderate to severe ulcerative colitis or who do not respond to 5-ASA drugs. Corticosteroids, also known as steroids, can be given orally, intravenously, through an enema, or in a suppository, depending on the location of the inflammation. These drugs can cause side effects such as weight gain, acne, facial hair, hypertension, mood swings, and an increased risk of infection. For this reason, they are not recommended for long-term use.
Immunomodulators—such as azathioprine and 6-mercapto-purine (6-MP) reduce inflammation by affecting the immune system. They are used for patients who have not responded to 5-ASAs or corticosteroids or who are dependent on corticosteroids. However, immunomodulators are slow-acting and may take up to 6 months before the full benefit is seen. Patients taking these drugs are monitored for complications including pancreatitis and hepatitis, a reduced white blood cell count, and an increased risk of infection. Cyclosporine A may be used with 6-MP or azathioprine to treat active, severe ulcerative colitis in people who do not respond to intravenous corticosteroids.

### Other drugs may be given to relax the patient or to relieve pain, diarrhea, or infection.

Occasionally, symptoms are severe enough that the person must be hospitalized. For example, a person may have severe bleeding or severe diarrhea that causes dehydration. In such cases the doctor will try to stop diarrhea and loss of blood, fluids, and mineral salts. The patient may need a special diet, feeding through a vein, medications, or sometimes surgery.

About 25-40% of ulcerative colitis patients must eventually have their colons removed because of massive bleeding, severe illness, rupture of the colon, or risk of cancer. Sometimes the doctor will recommend removing the colon if medical treatment fails or if the side effects of corticosteroids or other drugs threaten the patient's health. Surgery to remove the colon and rectum, known as proctocolectomy, is followed by one of the following:
Ileostomy, in which the surgeon creates a small opening in the abdomen, called a stoma, and attaches the end of the small intestine, called the ileum, to it. Waste will travel through the small intestine and exit the body through the stoma. The stoma is about the size of a quarter and is usually located in the lower right part of the abdomen near the beltline. A pouch is worn over the opening to collect waste, and the patient empties the pouch as needed.
Ileoanal anastomosis, or pull-through operation, which allows the patient to have normal bowel movements because it preserves part of the anus. In this operation, the surgeon removes the diseased part of the colon and the inside of the rectum, leaving the outer muscles of the rectum. The surgeon then attaches the ileum to the inside of the rectum and the anus, creating a pouch. Waste is stored in the pouch and passed through the anus in the usual manner. Bowel movements may be more frequent and watery than before the procedure. Inflammation of the pouch (pouchitis) is a possible complication.
   Not every operation is appropriate for every person. Which surgery to have depends on the severity of the disease and the patient's needs, expectations, and lifestyle. People faced with this decision should get as much information as possible by talking to their doctors, to nurses who work with colon surgery patients (enterostomal therapists), and to other colon surgery patients. Patient advocacy organizations can direct people to support groups and other information resources.

Most people with ulcerative colitis will never need to have surgery. If surgery does become necessary, however, some people find comfort in knowing that after the surgery, the colitis is cured and most people go on to live normal, active lives.

B. Crohn's Disease

As with ulcerative colitis, O-glycans have been suggested as playing a role in Crohn's disease, another inflammatory disease of the gastro-intestinal tract. Crohn's disease is characterized by intestinal inflammation and the development of intestinal stenosis and fistulas; neuropathy often accompanies these symptoms. One hypothesis for the etiology of Crohn's disease is that a failure of the intestinal mucosal barrier, possibly resulting from genetic susceptibilities and environmental factors (e.g., smoking), exposes the immune system to antigens from the intestinal lumen including bacterial and food antigens (e.g., Soderholm et al., 1999; Hollander et al., 1986; Hollander, 1992). Another hypothesis is that persistent intestinal infection by pathogens such as Mycobacterium paratuberculosis, Listeria monocytogenes, abnormal Escherichia coli, or paramyxovirus, stimulates the immune response; or alternatively, symptoms result from a dysregulated immune response to ubiquitous antigens, such as normal intestinal microflora and the metabolites and toxins they produce (Sartor, 1997). The presence of IgA and IgG anti-Sacccharomyces cerevisiae antibodies (ASCA) in the serum was found to be highly diagnostic of pediatric Crohn's disease (Ruemmele et al., 1998; Hoffenberg et al., 1999).

Recent efforts to develop diagnostic and treatment tools against Crohn's disease have focused on the central role of cytokines (Schreiber, 1998; van Hogezand & Verspaget, 1998). Cytokines are small secreted proteins or factors (5 to 20 kD) that have specific effects on cell-to-cell interactions, intercellular communication, or the behavior of other cells. Cytokines are produced by lymphocytes, especially T_{H}1 and T_{H}2 lymphocytes, monocytes, intestinal macrophages, granulocytes, epithelial cells, and fibroblasts (reviewed in Rogler &. Andus, 1998; Galley & Webster, 1996). Some cytokines are pro-inflammatory (e.g., TNF-α, IL-1(α and β), IL-6, IL-8, IL-12, or leukemia inhibitory factor (LIF)); others are anti-inflammatory (e.g., IL-1 receptor antagonist, IL-4, IL-10, IL-11, and TGF-β). However, there may be overlap and functional redundancy in their effects under certain inflammatory conditions.

In active cases of Crohn's disease, elevated concentrations of TNF-α and IL-6 are secreted into the blood circulation, and TNF-α, IL-1, IL-6, and IL-8 are produced in excess locally by mucosal cells (id.; Funakoshi et al., 1998). These cytokines can have far-ranging effects on physiological systems including bone development, hematopoiesis, and liver, thyroid, and neuropsychiatric function. Also, an imbalance of the IL-β/IL-1 ra ratio, in favor of pro-inflammatory IL-1β, has been observed in patients with Crohn's disease (Rogler & Andus, 1998; Saiki et al., 1998; Dionne et al., 1998; but see Kuboyama, 1998). One study suggested that cytokine profiles in stool samples could be a useful diagnostic tool for Crohn's disease (Saiki et al., 1998).

Anti-inflammatory drugs, such as 5-aminosalicylates (e.g., mesalamine) or corticosteroids, are typically prescribed, but are not always effective (reviewed in Botoman et al., 1998). Immunosuppression with cyclosporine is sometimes beneficial for patients resistant to or intolerant of corticosteroids (Brynskov et al., 1989). In Crohn's disease, a dysregulated immune response is skewed toward cell-mediated immunopathology (Murch, 1998). But immunosuppressive drugs, such as cyclosporine, tacrolimus, and mesalamine have been used to treat corticosteroid-resistant cases of Crohn's disease with mixed success (Brynskov et al., 1989; Fellerman et al., 1998). Nevertheless, surgical correction is eventually required in 90% of patients; 50% undergo colonic resection (Leiper et al., 1998; Makowiec et al., 1998). The recurrence rate after surgery is high, with 50% requiring further surgery within 5 years (Leiper et al., 1998; Besnard et al., 1998). Other therapies include the use of various cytokine antagonists (e.g., IL-Ira), inhibitors (e.g., of IL-1β converting enzyme and antioxidants) and anti-cytokine antibodies (Rogler and Andus, 1998; van Hogezand & Verspaget, 1998; Reimund et al., 1998; Lugering et al., 1998; McAlindon et al., 1998). Monoclonal antibodies against TNF-α have been tried with some success in the treatment of Crohn's disease (Targan et al., 1997; Stack et al., 1997; van Dullemen et al., 1995).

Another approach to the treatment of Crohn's disease has focused on at least partially eradicating the bacterial community that may be triggering the inflammatory response and replacing it with a non-pathogenic community. For example, U.S. Pat. No. 5,599,795 discloses a method for the prevention and treatment of Crohn's disease in human patients. Their method was directed to sterilizing the intestinal tract with at least one antibiotic and at least one anti-fungal agent to kill off the existing flora and replacing them with different, select, well-characterized bacteria taken from normal humans. Borody taught a method of treating Crohn's disease by at least partial removal of the existing intestinal microflora by lavage and replacement with a new bacterial community introduced by fecal inoculum from a disease-screened human donor or by a composition comprising Bacteroides and Escherichia coli species. (U.S. Pat. No. 5,443,826). However, there has been no known cause of Crohn's disease to which diagnosis and/or treatment could be directed.

### III. GASTROINTESTINAL CANCER

Circumstantial evidence has suggested that O-glycans may play a role in gastrointestinal cancers. The present inventors have found, surprisingly, that O-glycans can themselves prove inhibitory of such cancers. Thus, an O-glycan compound of the present invention (e.g., a mucin) is proposed here for the prevention and treatment of a gastrointestinal cancer, such as colorectal cancer. Gastrointestinal cancers that may be prevented or treated via the present invention include colorectal cancer (e.g., a colorectal adenoma, a colorectal carcinoma, or a colorectal adenomatous polyp), stomach cancer, a cancer of the large or small intestine, and esophageal cancer. In certain embodiments, the gastrointestinal cancer prevented or treated using the present invention is colorectal cancer.

Colorectal cancer is a term used to refer to cancer that starts in the colon or rectum. Colon and rectal cancers begin in the digestive system, also called the GI (gastrointestinal) system. This is where food is processed to create energy and rid the body of waste matter. Colorectal cancer primarily affects men and women aged 50 years or older. For men, colorectal cancer is the third most common cancer after prostate cancer and lung cancer. For women, colorectal cancer is the third most common cancer after breast cancer and lung cancer.

Cancer that starts in the different areas of the colon may cause different symptoms. In most cases, colon and rectum cancers develop slowly over a period of several years. Most of these cancers begin as a polyp—a growth of tissue into the center of the colon or rectum. A type of polyp known as adenoma can become cancerous. Removing the polyp early may prevent it from becoming cancer. Over 95% of colon and rectal cancers are adenocarcinomas. These are cancers of the cells that line the inside of the colon and rectum. There are some other, more rare, types of tumors of the colon and rectum.

Thus, the present invention involves the use of O-glycan compositions for the prevention, inhibition or treatment of cancers. Aqueous compositions of the present invention will have an effective amount of an O-glycan composition that inhibits, prevents or reducing the symptoms of a cancer. Such compositions will generally be dissolved or dispersed in a pharmaceutically acceptable carrier, diluent or aqueous medium. Various dosing regimens are contemplated, including every other day, once daily, twice daily, three times daily. Chronic, long-term administration also is contemplated for individuals at risk of developing gastointestinal cancer. Generally, oral administration will be the route of choice although intravenous or intramuscular injection may be utilized.

In terms of prevention, patients with ulcerative colitis appear to be at increased risk for colorectal cancer and thus constitute a class that may receive prophylactic/preventive administrations. Several factors have been suggested to be associated with a higher risk of colorectal cancer in patients with IBD. Increasing the duration of disease is generally accepted as a risk factor, with colorectal cancer rarely being diagnosed when ulcerative colitis has been present for less than 8 years. The age of onset has been suggested to be related to the risk of developing colorectal cancer. A family history of colorectal cancer is also a risk factor; patients with ulcerative colitis and Crohn's disease with a first-degree relative with colorectal cancer have a relative risk of 2.5 and 3.7, respectively, for developing colorectal cancer, and if the first-degree relative was diagnosed with colorectal cancer before age 50 years, the relative risk is 9.2.

The extent of the ulcerative colitis is also a risk factor for developing colorectal cancer in most studies. It has been reported that the incidence ratio for the risk of colorectal cancer in patients with proctitis is 1.7, for patients with disease extending beyond the rectum but no further than the hepatic flexure is 2.8, and for patients with disease beyond the hepatic flexure is 14.8.

### IV. COMBINATION THERAPIES

An O-glycan compound or composition (e.g., mucins) may be administered in combination with another agent for the treatment of a cancer (e.g., colorectal cancer) or an inflammatory bowel disease. By combining agents, an additive effect may be achieved while not increasing the toxicity (if any) associated with a monotherapy. In addition, it is possible that more than additive effects (“synergism”) may be observed. Thus, combination therapies are a common way to exploit new therapeutic regimens.

The O-glycan treatment may precede, be co-current with and/or follow the other agent(s) by intervals ranging from minutes to weeks. In embodiments where the O-glycan treatment and other agent(s) are applied separately to a cell, tissue or organism, one would generally ensure that a significant period of time did not expire between the time of each delivery, such that the O-glycan treatment and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) with the O-glycan treatment. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the O-glycan treatment.

Various combination regimens of the O-glycan treatment and one or more agents may be employed. Non-limiting examples of such combinations are shown below, wherein a O-glycan treatment is “A” and an agent is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

A. IBD Combination Therapies

The O-glycan therapies of the present invention can be used in conjunction with other therapies that are used for the treatment of IBD. Thus, one may use a O-glycan (e.g., a mucin) in combination with another agent used for treating IBD, as discussed below.

Traditionally, a stepwise approach to treatment of IBD is employed. The aminosalicylates/NSAIDS and symptomatic agents would be considered the first line of treatment. Next, one would turn to antibiotics particularly in persons with Crohn's Disease who have perianal disease or an inflammatory mass. Corticosteroids are the next line of defense, having more significant side effects that other anti-inflammatories. Immune modifying agents can be used if corticosteroids do not provide the desired results. Drugs from any of the foregoing categories may be used together. Some specific examples are provided below.

Aminosalicylates. Aminosalicylates are anti-inflammatory drugs in the aspirin family. There are five aminosalicylate preparations available for use in the United States: sulfasalazine (Azulfidine), mesalamine (Asacol, Pentasa), olsalazine (Dipentum), and balsalazide (Colazal). These drugs can be given either orally or rectally (enema, suppository formulations).

NSAIDS. Nonsteroidal anti-inflammatory agents (NSAIDs) work by inhibiting the production of prostaglandins. Non-limiting examples include, ibuprofen, ketoprofen, piroxicam, naproxen, naproxen sodium, sulindac, aspirin, choline subsalicylate, diflunisal, oxaprozin, diclofenac sodium delayed release, diclofenac potassium immediate release, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate.

Corticosteroids. Corticosteroids are powerful, fast-acting anti-inflammatory agents. Their use in IBD is for acute flare-ups only. Corticosteroids may be administered by a variety of routes, depending upon the location and severity of disease; they may be administered intravenously (methylprednisolone, hydrocortisone) in the hospital, orally (prednisone, prednisolone, budesonide, dexamethasone), or rectally (enema, suppository, foam preparations). Corticosteroids tend to provide rapid relief of symptoms as well as a significant decrease in inflammation, but their side effects limit their use (particularly longer-term use).

Immune modifiers. Immune modifiers include 6-mercaptopurine (6-MP, Purinethol) and azathioprine (Imuran). Immune modifiers may work by causing a reduction in the lymphocyte count (a type of white blood cell). They are often used when aminosalicylates and corticosteroids are either ineffective or only partially effective. They are useful in reducing or eliminating some patient's dependence on corticosteroids. Immune modifiers may also prove helpful in maintaining remission in some persons with refractory ulcerative colitis.

Anti-TNF agent. Infliximab (Remicade) is an anti-TNF agent, acting by binding to TNF, thereby inhibiting its effects on the tissues. It is approved by the FDA for the treatment of persons with moderate-to-severe Crohn's Disease who have had an inadequate response to standard medications. In such persons, a response rate of 80% and a remission rate of 50% have been reported.

Antibiotics. Metronidazole and ciprofloxacin are the most commonly used antibiotics in persons with IBD. Antibiotics are used sparingly in persons with ulcerative colitis because they have an increased risk of developing antibiotic-associated pseudomembranous colitis. In persons with Crohn's Disease, antibiotics are used for the treatment of complications (perianal disease, fistulae, inflammatory mass).

Symptomatic treatments. One can also provide antidiarrheal agents, antispasmodics, and acid suppressants for symptomatic relief.

B. Cancer Combination Therapies

In another embodiment, the present invention may be used to prevent or delay the development of a gastrointestinal cancer, or to reduce the symptoms thereof. There are few agents that may be used to prevent the development of cancer, although non-steroidal anti-inflammatory drugs (peroxicam, sulindac, aspirin) have been suggested to have preventative action with respect to colorectal cancer. Also, diets incorporating high fiber, fruits and vegetables also are associated with lowered colorectal cancer risk.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Mice that Lack Core 3-Derived O-Glycans are Much More Susceptible to DSS-Induced Colitis Compared to Wild-Type (WT) Littermates

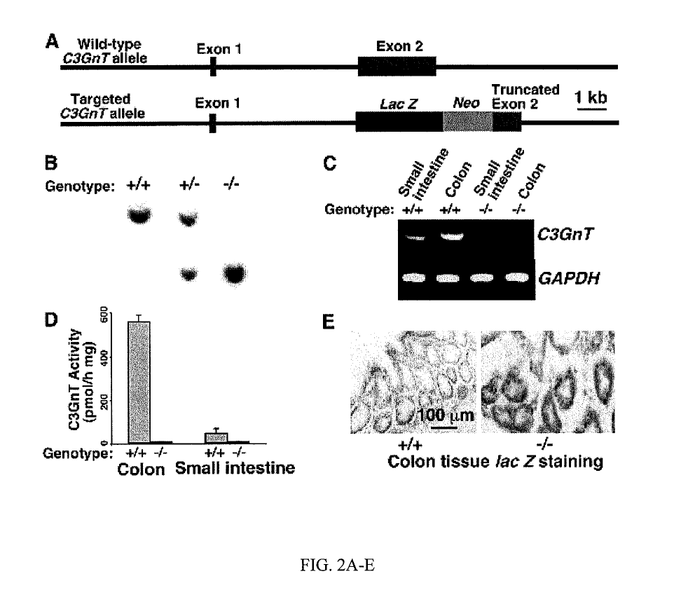

Mice that lack core 3-derived O-glycans are much more susceptible to DSS-induced colitis as compared to wild-type littermates. C3GnT, the key enzyme for the formation of core 3-derived O-glycans, is predominantly expressed in intestinal epithelia and especially in colonic tissue (Iwai et al., 2002 and data not shown). To study the role of core 3-derived O-glycans in intestinal function, the inventors established a conventional C3GnT gene-deficient mouse line (C3GnT^{-/-}) as illustrated in FIGS. 2A and 2B. The lac Z reporter was integrated immediately after the endogenous C3GnT promoter region to identify the expression pattern of C3GnT (FIG. 2A). RT-PCR and enzymatic assays revealed that the C3GnT mRNA transcription and enzyme activity in tissue extracts were eliminated in C3GnT^{-/-} mice (FIGS. 2C and 2D). Lac Z staining of different C3GnT^{-/-} tissues confirmed that expression of the C3GnT was restricted to intestine (FIG. 2E).

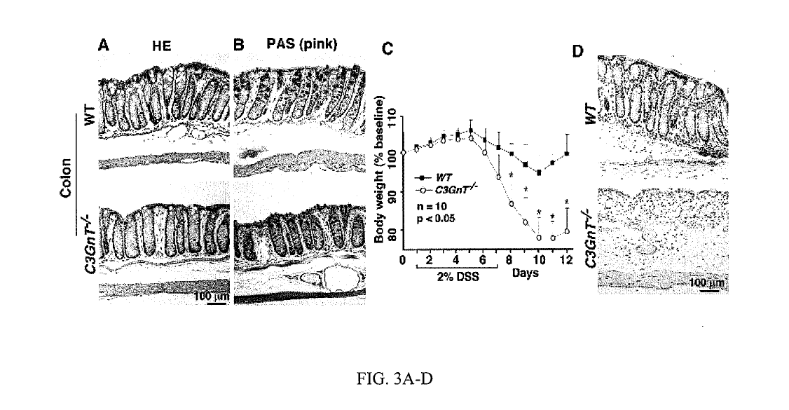

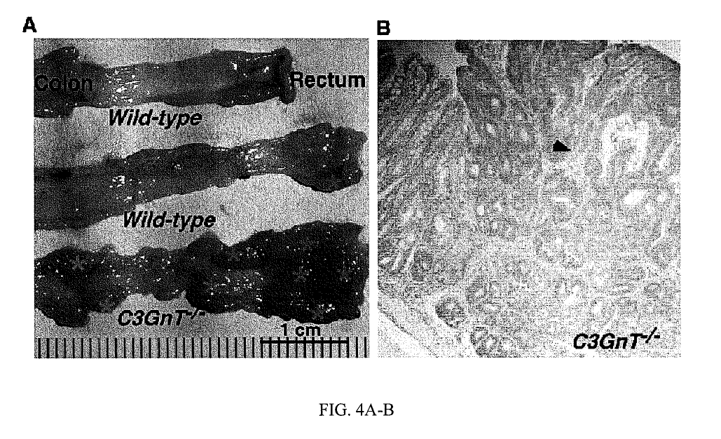

The C3GnT^{-/-} mice developed and bred normally. Gross morphology and histological examinations of multiple organs revealed no observable differences between WT and C3GnT^{-/-} mice (FIG. 3A). Periodic acid-Schiff (PAS) and an anti-murine Muc2 peptide antibody staining of intestinal tissues, however, demonstrated that the expression of carbohydrate moieties and Muc2, the major intestinal mucin, were significantly reduced in C3GnT^{-/-} colonic tissue (FIGS. 3B and 3C). To investigate the consequence of the altered glycosylation, the inventors challenged the six-week-old C3GnT^{-/-} males and WT males with 2% DSS (molecular weight 40 kDa, INC Biomedicals Inc.) in drinking water for 7 days, followed by 4 days of water without DSS (Stevceva et al., 2001). These experimental conditions induced a much more severe form of colitis in the C3GnT^{-/-} mice compared to WT, with much greater severity of weight loss, diarrhea, and fecal bleeding (FIG. 4A and data not shown). The inflammation was restricted to the colon, especially in the distal colonic region, while the small intestine was not significantly affected (FIG. 4B). These experiments demonstrate the significance of core 3-derived O-glycans in intestinal function.

Mice that lack intestinal core 1-derived O-glycans develop spontaneous ulcerative colitis. Core 1-derived O-glycans are a predominant form of O-glycans and are expressed in many tissues (Varki et al., 1999). To evaluate the role of core 1-derived O-glycans specifically in intestinal tissue, the inventors established a mouse line with an intestinal epithelial cell-specific deficiency of T-synthase (Epi T-syn^{-/-}), the key enzyme for the biosynthesis of core 1-derived O-glycans. This line was generated using the well-established Cre/loxP system.

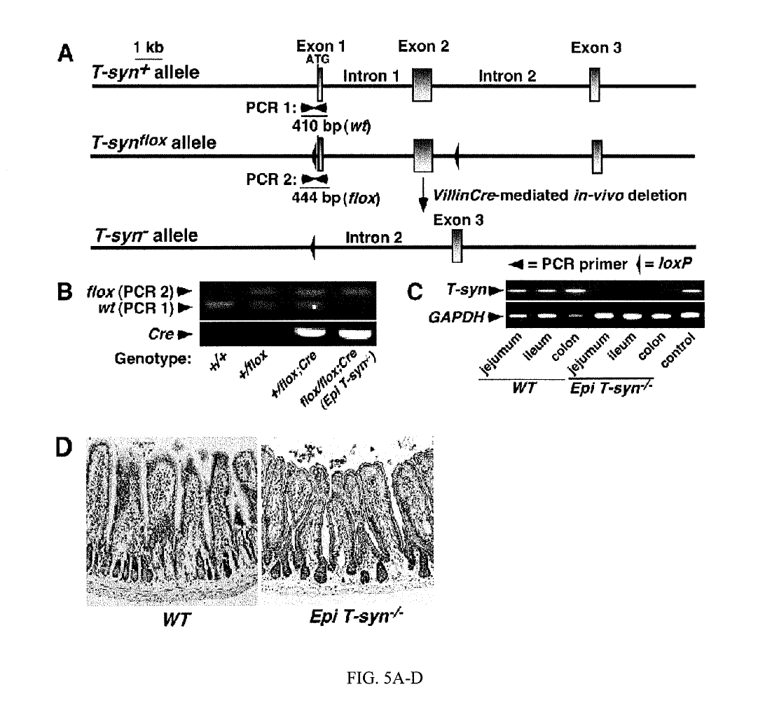

The inventors first developed mice in which the T-syn gene was flanked by loxP sites (T-syn^{flox/flox} mice, data not shown). To generate the Epi T-syn^{-/-} mice, the T-syn^{flox/flox} mice were bred with a transgenic line expressing Cre recombinase specifically in intestinal epithelial cells, under control of a Villin promoter (VillinCre Mice, Jackson Laboratories) (FIGS. 5A and 5B). The Epi T-syn^{-/-} mice were born in expected Mendelian ratios and displayed no phenotypic defects at birth. Characterization of T-syn transcripts by RT-PCR indicated that the VillinCre-mediated in vivo deletion of the floxed T-syn gene is complete in isolated epithelial cells (FIG. 5C). To determine the specificity of the in vivo deletion, the inventors probed Epi T-syn^{-/-} intestinal tissue sections with a monoclonal antibody (mAb) to Tn antigen. It was expected that WT tissue would not express Tn antigen. Specific VillinCre-mediated deletion was expected to abolish the synthesis of core 1-derived O-glycans and expose Tn antigen exclusively in the intestinal epithelial cells of Epi T-syn^{-/-} mice. As predicted, immunochemical staining with anti-Tn mAb did not label WT intestinal tissue. In contrast, anti-Tn mAb labeled Epi T-syn^{-/-} intestinal epithelial cells, but not other cell types in these mice (FIG. 5D). These observations verify the specificity of the VillinCre-mediated deletion.

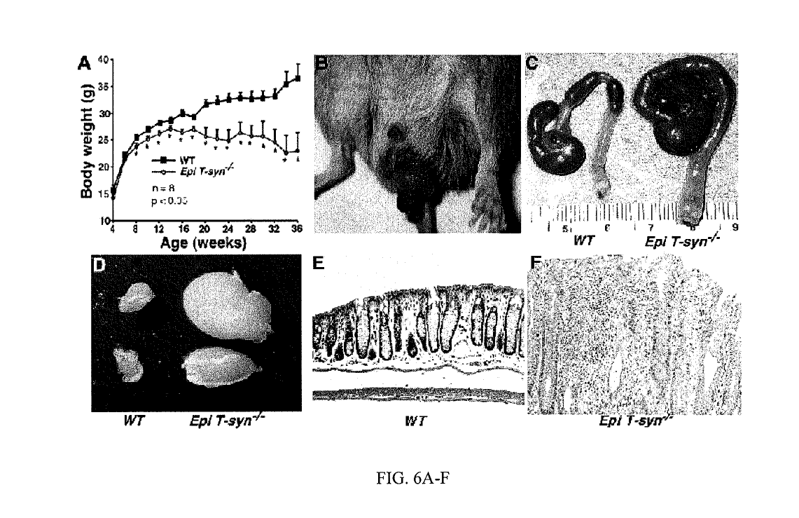

Young Epi T-syn^{-/-} mice, less than 5-weeks old, were indistinguishable from littermate controls. However, beginning at 6-weeks, about 20% of Epi T-syn^{-/-} mice displayed diarrhea, and at 12 weeks, all Epi T-syn^{-/-} mice had diarrhea and some had occasional bloody stool. Obvious weight loss was evident by 8 weeks in male Epi T-syn^{-/-} mice (FIG. 6A). About 15% of Epi T-syn^{-/-} mice had rectal prolapse (FIG. 6B), and the disease severity progressed over time. The colon of Epi T-syn^{-/-} mice, especially the distal colon and rectum, had dilated and thickened walls (FIG. 6C). In fact, this region always displayed disease and was always the most severely affected. Enlarged mesenteric lymph nodes (MLN) were common (FIG. 6D). Microscopic examination showed no abnormalities in major organs such as heart, liver, stomach, spleen and thymus. However, Epi T-syn^{-/-} colon exhibited significant inflammation, characterized by epithelial ulceration, inflammatory cell infiltration, goblet cell loss, epithelial hyperplasia, and frequently, crypt microabscess (FIGS. 6E and 6F).

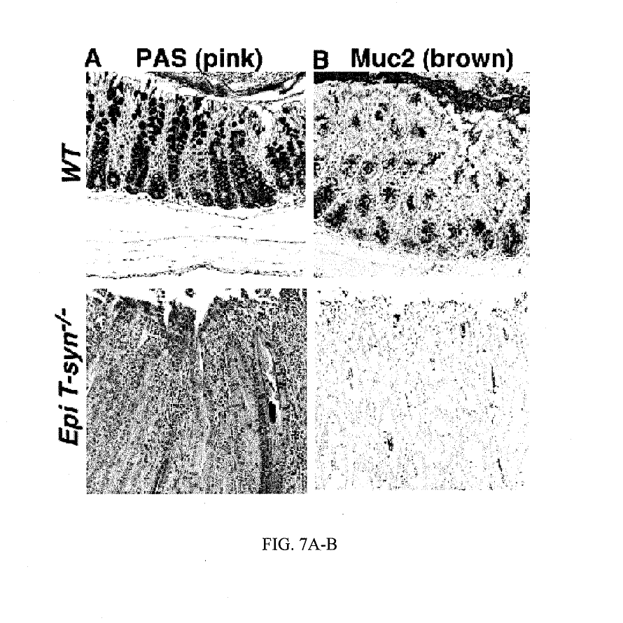

PAS and Muc2 staining revealed that Epi T-syn^{-/-} mice had significantly reduced carbohydrates, especially the mucus layer and Muc2 staining (FIGS. 7A and 7B). Moreover, HPLC analysis of Epi T-syn^{-/-} colonic mucosa showed a dramatic decrease in O-glycan quantity and diversity compared to WT colonic mucosa (FIG. 7C). Thus the loss of O-glycans correlates with the pathology of colitis in Epi T-syn^{-/-} mice.

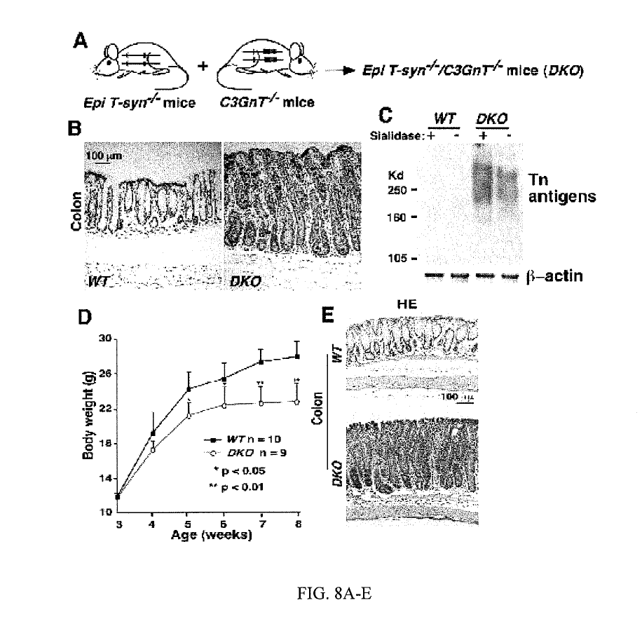

Mice lacking both core 1- and 3-derived O-glycans display severe ulcerative colitis that is spontaneous and has an early onset. Core 1- and core 3-derived O-glycans are the predominant glycan components of intestinal mucus (Corfield et al., 2001; Varki et al., 1999). To study their roles, the inventors established mice lacking core 3-derived O-glycans as well as intestinal core 1-derived O-glycans (DKO). DKO mice were generated by cross breeding Epi T-syn^{-/-} and C3GnT^{-/-} mice (FIG. 8A). Immunohistochemical staining with an anti-Tn mAb in the DKO colonic tissue, and Western blotting of colonic tissue extracts with a Tn-specific lectin, HPA, revealed Tn antigen expression in the DKO colonic tissue but not in the WT tissue (FIGS. 8B and 8C). Sialidase treatment did not significantly alter the Tn staining pattern, suggesting that most of the Tn antigens were not capped by sialic acids (FIG. 8C). Staining with PAS confirmed the dramatic reduction of carbohydrates in DKO colonic tissues (FIG. 8D). Weak residual staining of PAS in DKO tissue was observed and may reflect staining of Tn antigens. Decreased Muc2 staining in DKO tissues suggests that the absence of O-glycans affects expression of this mucin (FIG. 8E).

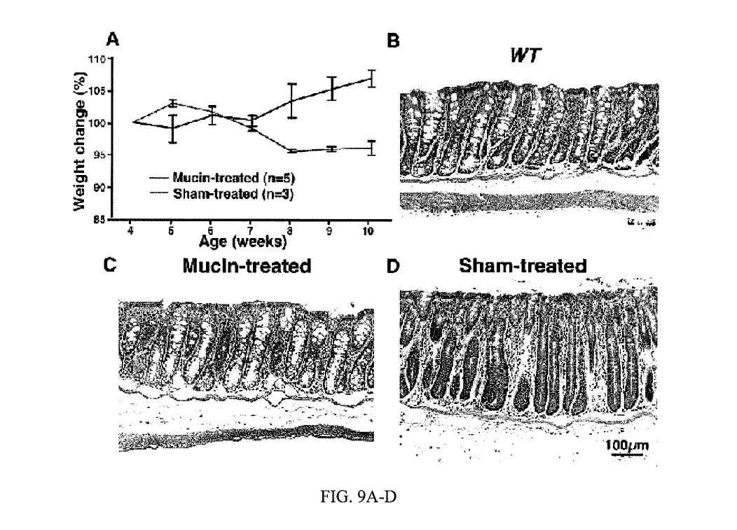

DKO mice developed an early onset form of spontaneous ulcerative colitis that was much more severe than that of Epi T-syn^{-/-} mice. The DKO mice developed disease as early as 3 weeks after birth (data not shown), and the severity of the disease continued to progress with time (FIGS. 9A-D). Like Epi T-syn^{-/-} mice, inflammation was restricted primarily to the distal colon, which mimics the human disease.

### Example 2—Oral Administration of Mucins Prevents Ulcerative Colitis in Epi T-syn^{-/-} Mice

Method for exogenous mucin preparation. The inventors purified mucins from fresh porcine stomach or colon according to published methods with modifications (Xia et al., 2005; Feste et al., 1990). Briefly, after removing contents and briefly rinsing in water, the mucosal layer (including epithelium and mucus) of porcine stomach or colon was removed by scraping. The mucosal material is homogenized in ice-cold water (˜1 part mucosa: 1 part water, final slurry), and centrifuged to remove insoluble debris. The soluble mucins in the supernatant were precipitated by adjusting to pH 5.0 with 100 mM HCl followed by centrifugation (10,000×g, 4° C., 10 min). The pellet was resolubilized and adjusted to pH 7.2 with 100 mM NaOH, then extracted twice in methanol:chloroform (1:1 v/v) prior to a second centrifugation. The middle phase was collected and dialyzed (12-14,000 MWCO) followed by sequential treatment with heparinum Heparinase II (0.075 U/ml, Sigma), chondroitinase ABC (0.015 U/ml, Sigma), DNase (75 U/ml, Invitrogen), RNase (0.01 mg/ml, Invitrogen), and proteinase K (0.25 U/ml, O/N at 65° C., Sigma). These treatments eliminate contaminating lipids, polypeptides, and nucleotides. The mucin was then collected as a >200 kDa void volume fraction by size exclusion chromatography (Sephacryl HR-S-200, Pharmacia) in isotonic buffer (50 mM Tris, 100 mM NaCl, pH 7.4). The void volume fraction was dialyzed, lyophilized, weighed, and stored at −80° C. The quality of the purified mucins was verified by SDS-PAGE using a 3% stacking and a 4% separating gel that is stained by PAS. Protein was measured using a BCA kit (Pierce).

Oral administration of exogenous mucin in EPI T-SYN^{-/-} mice. The inventors' experiments demonstrated that O-glycan-deficient mice showed a significant reduction in Muc2 staining, a reduced intestinal mucus gel layer, and high susceptibility to ulcerative colitis. Because secretory mucins are the major components of the intestinal mucus gel layer, they reasoned that administration of exogenous mucins might be therapeutic. The inventors therefore tested whether oral administration of purified porcine stomach mucins could prevent ulcerative colitis in Epi T-syn^{-/-} mice. Epi T-syn^{-/-} mice were treated with 50 mg mucin/mouse/day in Napa-Nector, a commonly used hydration source. The control Epi T-syn^{-/-} mice were treated with Napa-Nector only. Mice were treated for seven weeks beginning at 4-weeks old. Treated mice developed a much less severe form of colitis compared to the untreated controls. Although preliminary, the experiment suggests that loss of O-glycans primarily affects the function of the mucus gel layer, and exogenous mucins are of great potential therapeutic value for the treatment of ulcerative colitis (FIGS. 9A-D).

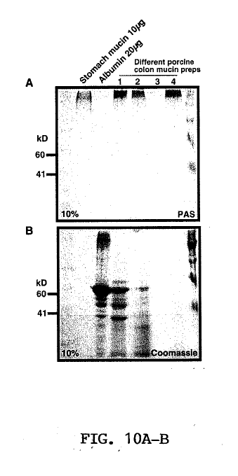

Preparation of mucins. The inventor also investigated whether mucins isolated from porcine colon as opposed to stomach produce a better therapeutic effect. In the experiment described above, the inventor used porcine stomach mucins. Although this reagent exhibited preventive effect against colitis in our model, he sought to determine whether mucins from porcine colon would have a better therapeutic effect versus the stomach mucins against colitis. Therefore, mucins were purified from fresh porcine colon. Briefly, after removing contents and briefly rinsing in water, the mucosal layer (including epithelium and mucus) of porcine colon was removed by scraping. The mucosal material was homogenized in ice-cold water (1 part mucosa: 1 part water, final slurry), and centrifuged to remove insoluble debris. The soluble mucins in the supernatant were precipitated by 75% alcohol followed by centrifugation (10,000×g, 4° C., 10 min). The pellet was resolubilized, dialyzed, lyophilized, weighed, and stored at −80° C. The quality of the purified mucins was verified by SDS-PAGE, and was stained by PAS (FIGS. 10A-B).

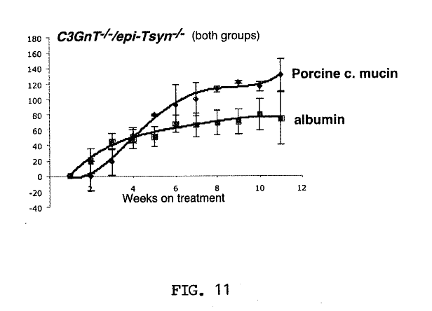

C3GnT−/−/Epi T-syn^{-/-} mice (6-weeks old) were divided into three groups (3 mice per group). The first group was fed purified porcine colon mucins mixed in Napa-Nector, at 50 mg mucins/mouse/day. The second group will be on Napa-Nector containing the same amount (20% of the mucin weight) of albumin. Preliminary data showed that porcine colon mucins treated mice gained weight much faster than controls (FIG. 11). Although preliminary, these experiments suggests that loss of O-glycans primarily affects the function of the mucus gel layer, and exogenous mucins are of great potential preventive and therapeutic values for ulcerative colitis.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 5,440,013
U.S. Pat. No. 5,443,826
U.S. Pat. No. 5,446,128
U.S. Pat. No. 5,475,085
U.S. Pat. No. 5,599,795
U.S. Pat. No. 5,618,914
U.S. Pat. No. 5,670,155
U.S. Pat. No. 5,672,681
U.S. Pat. No. 5,674,976
U.S. Pat. No. 5,710,245
U.S. Pat. No. 5,840,833
U.S. Pat. No. 5,859,184
U.S. Pat. No. 5,929,237
Besnard et al., Gut, 43(5):634-638, 1998.
Botoman et al., Am. Fam. Physician, 57(1):57-68, 1998.
Brynskov et al., N. Engl. J. Med., 321(13):845-850, 1989.
Byrd and Bresalier, Cancer Metastasis Rev., 23(1-2):77-99, 2004.
Corfield et al., Front Biosci., 6:D1321-1357, 2001.
Dionne et al., Clin. Exp. Imunol., 112(3):435-442, 1998.
Dixon et al., Postgrad Med J., 82(963):76-78, 2006.
Elson et al., Inflamm. Bowel Dis., 9:137-153, 2003.
Fellerman et al., Am. J. Gastroenterol., 93(10):1860-1866, 1998.
Feste et al., J. Chromatogr., 518:349-359, 1990.
Funakoshi et al., Digestion, 59(1):73-78, 1998.
Galley and Webster, Br. J. Anaesth., 77:11-16, 1996.
Hoffenberg et al., J. Pediatr., 134(4):447-452, 1999.
Hollander et al., Ann. Intern. Med., 105:883-885, 1986.
Hollander, Scand. J. Gastroenterol., 27:721-726, 1992.
Iwai et al., J. Biol. Chem., 277:12802-12809, 2002.
Johannesson et al., J. Med. Chem., 42(4):601-608, 1999.
Johnson et al., In: Biotechnology And Pharmacy, Pezzuto et al. (Eds.), Chapman and Hall, NY, 1993.
Kuboyama, Kurume Med. J, 45(1):33-37, 1998.
Kyte and Doolittle, J. Mol. Biol., 157(1):105-132, 1982.
Leiper et al., Baillieres Clin. Gastroenterol., 12(1):179-199, 1998.
Lugering et al., Ital. J. Gastroenterol. Hepatol., 30(3):338-344, 1998.
Makowiec et al., Z. Gastroenterol., 36(8):619-624, 1998.
McAlindon et al., Gut, 42(2):214-219, 1998.
Murch, Nutrition, 14:780-783, 1998.
Podolsky and Fournier, Gastroenterology, 95:379-387, 1988.
Podolsky Isselbacher, Gastroenterology, 87:991-998, 1984.
Podolsky, N. Engl. J. Med., 347:417-429, 2002.
Reimund et al., Eur. J. Clin. Invest., 28(2):145-150, 1998.
Remington's Pharmaceutical Sciences, 18th Ed. Mack Printing Company, pp. 1289-1329, 1990.
Rhodes, Gut., 40:807-808, 1997.
Rhodes, Lancet., 347:40-44, 1996.
Rogler and Andus, World J. Surg., 22(4):382-389, 1998.
Ruemmele et al., Gastroenterol., 115(4):822-829, 1998.
Saiki et al., Scand. J. Gastroenterol., 33(6):616-622, 1998.
Sartor, Am. J. Gastroenterol., 92(12):5S-11S, 1997.
Sartor, Curr. Opin. Gastroenterol., 19:358-365, 2003.
Schreiber, Neth. J. Med., 53(6):S24-31, 1998.
Soderholm et al., Gastroenterol., 117:65-72, 1999.
Stack et al., Lancet, 349(9051):521-524, 1997.
Stevceva et al., BMC Clin. Pathol., 1:3, 2001.
Targan et al., N. Engl. J. Med., 337(15):1029-1035, 1997.
van Dullemen et al., Gastroenterol., 109(1):129-135, 1995.
van Hogezand and Verspaget, Drugs, 56(3):299-305, 1998.
Varki et al., In: Essentials of Glycobiology, Cold Spring Harbor Press, Cold Spring Harbor, N.Y., 1999.
Vita et al., Biopolymers, 47:93-100, 1998.
Weisshoff et al., Eur. J. Biochem., 259(3):776-788, 1999.
Xia et al., Glycobiology, 15:747-775, 2005.

1. A method of preventing development of or treating an inflammatory bowel disease comprising administering to a subject in need thereof an O-glycan composition.

2. The method of claim 1, wherein the inflammatory bowel disease is ulcerative colitis.

3. The method of claim 1, wherein the inflammatory bowel disease is Crohn's disease.

4. The method of claim 1, wherein the O-glycan composition comprises a mucin composition.

5. The method of claim 4, wherein the mucin composition comprises one or more of Muc1, Muc2, Muc3, Muc4, Muc5AC, Muc6, or Muc13.

6. The method of claim 1, further comprising administering to said subject a second therapeutic composition.

7. The method of claim 6, wherein the second therapeutic composition is an anti-inflammatory agent or an antibiotic.

8. The method of claim 1, wherein the O-glycan composition is formulated for release in the stomach.

9. The method of claim 1, wherein the O-glycan composition is formulated for release in the small intestine.

10. The method of claim 8, wherein the O-glycan composition is formulated for release in the ileum, jejunum or duodenum.

11. The method of claim 1, wherein the O-glycan composition is formulated for release in the large intestine.

12. The method of claim 9, wherein the O-glycan composition is formulated for release in the cecum, ascending colon, transverse colon, descending colon, sigmoid colon or rectum.

13. The method of claim 1, wherein said subject is a mammal.

14. The method of claim 1, wherein said subject is a human.

15. The method of claim 4, wherein the mucin composition comprises mucins obtained from a mammal.

16. The method of claim 14, wherein said mucins are purified by centrifugation.

17. The method of claim 14, wherein said mucins are treated with DNAse, RNAse, protease and lipase.

18. The method of claim 15, wherein said mucins are further purified by chromatography.

19. The method of claim 14, wherein the mucins are derived from stomach or colon.

20. The method of claim 14, wherein the mucins are human mucins.

21. The method of claim 14, wherein the mucins are non-human mucins.

22. The method of claim 4, wherein said mucins are recombinantly expressed in a mammalian expression system.

23-29. (canceled)

30. A method of preventing development of colorectal tumor comprising administering to a subject in need thereof an O-glycan composition.

31. The method of claim 30, wherein the colorectal tumor is a colorectal adenomatous polyp.

32. The method of claim 30, wherein the colorectal tumor is colorectal adenoma.

33. The method of claim 30, wherein the colorectal tumor is colorectal carcinoma.

34. The method of claim 30, wherein the O-glycan composition comprises a mucin composition.

35. The method of claim 34, wherein the mucin composition comprises one or more of Muc1, Muc2, Muc3, Muc4, Muc5AC, Muc6, or Muc13.

36. The method of claim 30, further comprising administering to said subject a second therapy.

37. The method of claim 36, wherein the second anti-cancer therapy is such as an anti-inflammatory agent or an antibiotic.

38. The method of claim 30, wherein the O-glycan composition is formulated for release in the large intestine.

39. The method of claim 38, wherein the O-glycan composition is formulated for release in the cecum, stomach, ascending colon, transverse colon, descending colon, sigmoid colon or rectum.

40. The method of claim 30, wherein said subject is a mammal.

41. The method of claim 30, wherein said subject is a human.

42. The method of claim 34, wherein the mucin composition comprises mucins obtained from a mammal.

43. The method of claim 42, wherein said mucins are purified by centrifugation.

44. The method of claim 42, wherein said mucins are treated with DNAse, RNAse, protease and lipase.

45. The method of claim 44, wherein said mucins are further purified by chromatography.

46. The method of claim 42, wherein the mucins are derived from stomach or colon.

47. The method of claim 42, wherein the mucins are human mucins.

48. The method of claim 42, wherein the mucins are non-human mucins.

49. The method of claim 42, wherein said mucins are recombinantly expressed in a mammalian expression system.

50. A pharmaceutical composition comprising an O-glycan composition dispersed in a pharmaceutically acceptable buffer, diluent or excipient.

51. The composition of claim 50, wherein the O-glycan composition comprises a mucin composition.

52. The composition of claim 50, wherein the mucin composition comprises one or more of Muc1, Muc2, Muc3, Muc4, Muc5AC, Muc6, or Muc13.

53. The composition of claim 50, wherein the pharmaceutical composition is formulated for release in the small intestine.

54. The composition of claim 53, wherein the pharmaceutical composition is formulated for release in the ileum, jejunum or duodenum.

55. The composition of claim 50, wherein the pharmaceutical composition is formulated for release in the large intestine.

56. The composition of claim 54, wherein the pharmaceutical composition is formulated for release in the cecum, ascending colon, transverse colon, descending colon, sigmoid colon or rectum.

57. The composition of claim 50, wherein the pharmaceutical composition is formulated for release in the stomach.
